(19) United States
(12) Patent Application Publication
(10) Pub. No.: US 2025/0302379 A1
(43) Pub. Date: Oct. 2, 2025

(54) DEVICE, SYSTEM AND METHOD OF MONITORING AND ADMINISTERING THERAPY FOR POSITIONAL SLEEP APNEA

(71) Applicant: Michael Markus, Harleysville, PA (US)

(72) Inventor: Michael Markus, Harleysville, PA (US)

(21) Appl. No.: 19/091,312

(22) Filed: Mar. 26, 2025

Related U.S. Application Data
(60) Provisional application No. 63/570,145, filed on Mar. 26, 2024.

(51) Int. Cl. A61B 5/00; A61B 5/11

(52) U.S. Cl. CPC A61B 5/4818; A61B 5/1116; A61B 5/1126; A61B 5/6802; A61B 5/746; A61B 2560/0462

(57) ABSTRACT

Provided is a system and method for managing positional sleep apnea therapy comprising a garment that, in a first configuration, comprises a position control member that interferes with the ability of a patient wearing the garment in the first configuration to sleep in a supine position. A sensing unit cooperates with the garment in the first configuration and at least one of: (i) the garment in a second configuration or a second garment that lacks the position control member, and (ii) an article of clothing or an accessory that is worn by the sleeping patient. The sensor unit detects when the patient is sleeping in the supine position, and comprises a proximity sensor that detects the presence of the patient adjacent to the sensing unit. An alert engine issues an alert to the patient in response to the sensing unit detecting that the patient is sleeping in the supine position.

### CROSS-REFERENCE TO RELATED APPLICATIONS

This application claims the benefit of U.S. Provisional Application No. 63/570,145, filed Mar. 26, 2024, which is incorporated in its entirety herein by reference.

### BACKGROUND OF THE INVENTION

### 1. Field of the Invention

This application relates generally to a method and apparatus for treating positional obstructive sleep apnea and, more specifically, to a device, system and method for modifying a person's habitual sleeping position by sensing that the person is sleeping in a supine position and, in response, generating feedback that encourages the person to adjust their sleeping position to a position that at least partially mitigates an effect of sleep apnea.

### 2. Description of Related Art

Obstructive sleep apnea (OSA) negatively impacts millions of people around the world. Those afflicted with OSA experience breathing disruptions while sleeping when their throat muscles relax and block their airway. Such breathing disruptions are referred to herein as apneic events. Short-term effects include fatigue during the day from inadequate sleep as a result of being unable to sleep for sustained periods during the night. Longer term effects can include increased risk of conditions such as high blood pressure, heart problems, recurrent heart attack, stroke, irregular heartbeats, type 2 diabetes, metabolic syndrome, and liver problems.

Continuous positive airway pressure (“CPAP”) therapy is currently the gold standard, but many struggle with compliance. For example, CPAP therapy requires the user to wear a mask while sleeping. It is believed the mask causes discomfort to some users, keeping them awake at night and eventually causing them to stop using the CPAP machine in an effort to improve their comfort level and fall asleep. Further, CPAP therapy itself is an end treatment, requiring use for the duration of the user's affliction with OSA unless the user seeks an alternative therapy or addresses factors that contribute to the user's OSA condition (e.g., losing weight). Often this means that CPAP users will be required to utilize CPAP therapy for the remainder of their lives.

It is believed that a significant portion of individuals who suffer from OSA can be categorized as “positional.” Those who suffer from “positional” OSA (“POSA”) are believed to experience a greater number of, and/or more-severe apneic events while sleeping in the supine position than they would otherwise experience sleeping on their side, or in the prone position. CPAP therapy for such individuals would help to improve their sleep even while in the supine position, but such therapy does not correct the sleeping position as the underlying cause of apneic events for those individuals. Thus, the cost of CPAP therapy may be unnecessarily incurred without treating one of the root causes of apneic events suffered by those with POSA.

Attempts to treat those with POSA have utilized a system that provides feedback when a person rolls onto their back and is believed to be sleeping in the supine position. Such systems assume that a person laying in a horizontal orientation, accompanied by a lack of movement of the person means that the person is sleeping soundly, without experiencing apneic events. But the person may have removed the device that monitors sleeping position for any number of reasons such as discomfort, for example, after turning it on. As a result, the person appears to be in compliance with the therapy program but is actually not receiving the therapy at all, and therefore not receiving the intended benefits of the therapy.

Further, traditional therapies for sleep apnea require patients to wear a mask or a device a certain way, or on a certain part of the body to comply with the therapy. However, every patient is different and has different preferences where wearing the device is comfortable. Some patients may experience discomfort and prefer to wear the mask/device at another location, or in a different manner than that required by the therapy. For such patients a therapy that may otherwise be beneficial to the patient may not be effective due to a rigid requirement to wear a device/mask at a specific location while sleeping.

### BRIEF SUMMARY OF THE INVENTION

Accordingly, there is a need in the art for a device, system and method for treating patients with POSA. Such a device, system and method can detect actual compliance with a prescribed therapy, and/or allow patients to tailor placement of a device without negatively affecting the results of the therapy.

According to one aspect, the subject application involves a proximity sensor that is operable to detect the presence of a patient wearing a device associated with a POSA therapy. After sensing movement of the patient, the proximity sensor is activated to detect the presence of the patient. If the patient is detected, a sleeping position of the patient detected by a position sensor can be stored in a memory of the device, or in a memory provided to a network-connected storage device such as a server, from where the stored data can be retrieved to be displayed by an application on a mobile communication device, a network-connected computer terminal, etc. If the patient is not detected by the proximity sensor, a condition that may occur if the patient places the device on a nightstand instead of wearing it, any data collected by the position sensor can be discarded without saving it in the memory, designated as being potentially misleading, etc.

According to another aspect, the subject application involves an application to be used with a position sensor worn by a patient while sleeping during POSA therapy. In the application, the patient can be presented with an option to specify where on the patient's body the position sensor will be worn while the patient sleeps. For example, the patient may prefer to clip the position sensor to a portion of a waistband at the bottom of the patient's back, or on a belt or strap wrapped around the patient's torso in front of the patient's chest. By specifying where the position sensor is to be worn, the present system can relate movements of the position sensor to an adjustment of the patient's sleeping position.

According to one aspect, the subject application involves an adaptable therapy device that senses a position of a sleeping patient, and adjusts a response to the patient sleeping in an undesirable position, such as the supine position for example. According to such aspects, the therapy device can include an actuator that vibrates when the patient is detected to be sleeping in the supine position. For patients that regularly respond to vibrations, the magnitude of the vibration can be adjusted to a relatively-low setting in an effort to minimize disruptions to the patient's sleep. In contrast, for patients who fail to respond, or only occasionally respond to vibrations, the magnitude of the vibration can be set to a relatively-high level to promote compliance with the therapy by urging the patient to move out of the supine position.

According to another aspect, the subject application involves a therapy device including a position sensor that senses a position of a patient while sleeping and an actuator that provides physical stimulation causing the patient to move out of the supine position. The therapy device is used in combination with an implantable device that stabilizes the patient's throat while sleeping by providing gentle stimulation to throat muscles, thereby allowing the airway to remain open during sleep. Data collected by the therapy device, such as sleeping positions throughout the night along with data indicating whether physical and/or audible feedback was issued for example, can be time stamped and used to evaluate the efficacy of the implantable device by comparing such data before and after the implantation. The therapy device can also urge the patient toward sleeping positions that mitigate the onset of POSA events, thereby limiting the burden on the implantable device.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 shows an illustrative embodiment of a system for implementing a progressive POSA therapy program, the system including a garment with a position control member and sensor unit during Stage 1, a belt that lacks a position control member with sensor unit during Stage 2, a personal garment with sensor unit during Stage 3, and sound-based monitoring during Stage 4;

FIG. 2 is a flow diagram showing an illustrative embodiment of a method of using a configurable system as part of a progressive POSA therapy program; and

FIG. 3 is a flow diagram schematically depicting a method of encouraging a patient to sleep in a position other than the supine position according to some embodiments of a monitoring system that does not use a sensor physically coupled to the patient or a mechanized respiratory therapy (e.g., CPAP machine)

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

Some embodiments of the present technology include a multi-stage positional therapy system and method designed to promote sustainable sleep-behavior change. Combining a wearable device and sensor technology, the present technology initially provides support by interfering with the patient laying in the supine sleeping position by supporting an object adjacent to the spine of the patient. The present technology steadily reduces the reliance on physical-restrictions on the patient's sleeping position based on patient progress as determined by a sensing unit. Sleep position is monitored along with adherence data, and feedback is tailored based on the patient's progress. For example, the prompting device configuration can be changed at pre-defined milestones, optionally in an effort to gradually wean the patient off of, or at least lessen the patient's reliance on artificial POSA support relative to a state prior to beginning the present POSA therapy. This novel, data-driven approach aims to personalize POSA therapy and enhance long-term treatment outcomes.

Existing positional sleep apnea therapies lack adaptable support. To Summarize exemplary embodiments described herein, the present technology utilizes real-time sleep position monitoring and integrates individualized feedback mechanisms. Sensor data is analyzed to tailor at least one of vibration and auditory cues issued by a monitoring device or remote device, for example, to optimize progression through support-reduction stages. Progress metrics can be processed and visualized within a companion application executed by a computing system such as a mobile telephone device, desktop or notebook computer, etc. The system and method further allow for clinician oversight through a secure data portal, via which the patient's treating physician or another party authorized by the patient can access data relating to the patient's progress. This design offers an innovative, evidence-based approach to POSA treatment, prioritizing user engagement and adaptability for maximizing compliance.

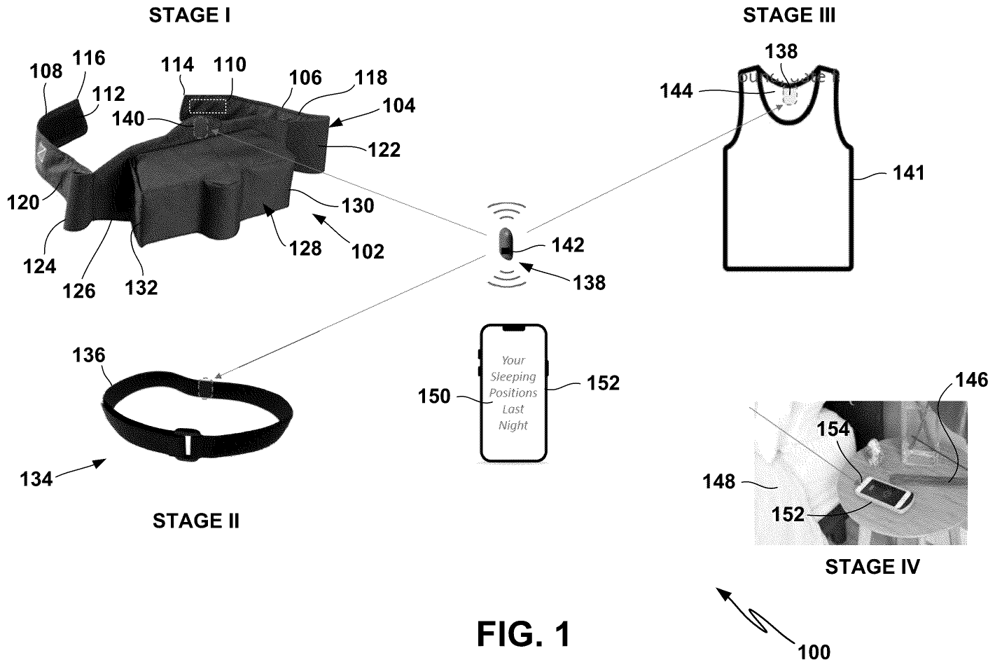

FIG. 1 shows an illustrative embodiment of system 100 for providing multi-stage POSA therapy. At least two, and optionally each of the stages of POSA therapy can offer different degrees of physical interference with the patient's ability to roll into the supine position. For example, Stage I therapy involves a garment 102 worn by a patient while the patient sleeps. The garment 102 can be specifically configured for interfering with the ability of a patient wearing the garment 102 to roll into the supine position. An example of the garment 102 can be found in U.S. Pat. No. 8,356,602 to Crocetti, which is incorporated in its entirety herein by reference. Generally, the garment 102 includes a belt 104 having first strap portions 106, 108 intended to encircle the side and front regions of a patient's torso. The strap portions 106, 108 include cooperating hook-and-loop (e.g., Velcro brand name) fastening elements 110, 112 adjacent distal edges 114, 116 thereof to maintain the belt 104 in a desired position/orientation about the patient's torso. The proximate regions 118, 120 of the belt 104 can be connected to intermediate, elastic sections 122, 124, to enable the belt 104 to be stretched about the wearer's torso prior to interconnecting the hook-and-loop fastening elements 110, 112. The resulting tension on the belt 104 imparted by the elastic forces from the elastic sections 122, 124 can maintain the garment 102 in a secure manner about the wearer's torso, and permit inspiratory chest excursion.

The illustrative embodiment of the garment 102 also includes a back panel 126 secured between the elastic sections 122, 144, and this back panel 126 includes a position control member 128 on (or in) it. As illustrated, the back panel supports an elongate, position control member 128 made of any suitable material, such as a material that is compressible to avoid causing a rigid object from poking the patient while sleeping, causing the patient to be awakened. Specific examples of the material can include a plastic foam (polyurethane, polystyrene, etc.). Outer edge surfaces 130, 132 are designed to engage the sleeping surface of a bed while the patient wearing the garment 102 is lying in a lateral position, i.e., lying on his/her side. This engagement aids in maintaining the individual in a lateral position to minimize the likelihood of the individual rolling into a supine position that could completely or partially close off the airway of the individual during the time he or she is sleeping.

According to some embodiments, the system 100 can include a belt 134 to be utilized during Stage II of a progressive POSA therapy program. The belt 134 can optionally be configured as belt 104 with the removable position control member 128 removed. According to alternate embodiments, however, the belt 134 can be a strip of material 136 comprising at least one elastic region, and can be smaller than the belt 134 used to support the position control member 128. For example, the strip of material 136 forming a substantial portion of the belt 134 can have a width from approximately one (1) inch wide to approximately six (6) inches wide, and the width can optionally be substantially constant along the length of the strip of material. Generally, the belt 134 is designed to be less bulky and, accordingly, less intrusive to wear than the garment 102. Further, the belt 134 lacks the position control member that forms an obstacle that interferes with the patient rolling into the supine position.

The system also includes sensor unit 138 that monitors a sleeping position of the patient wearing it. For example, sensor unit 138 can include electronic circuitry 140 configured as at least one of: (i) a three-axis accelerometer (or other position sensor), (ii) a Bluetooth (or other wireless transmission technology) transmitter, (iii) an alert engine that can issue vibratory and/or audible alerts, (iv) a timer circuit, and (v) a non-transitory computer-readable/writable medium such as solid state flash memory (a “data store”).

The sensor unit 138 is compact, and designed to be received within a pocket 140 formed in back panel 126 of the garment 102, clipped onto belt 104, belt 134, or a personal garment 141 such as an article of clothing (e.g., shirt, shorts, pajamas, undergarment, etc.). For example, during Stage III of the progressive POSA therapy program described herein, the sensor unit 138 can be clipped to a back neck region 144 of a shirt worn by the patient as the personal garment 141 while sleeping. The personal garment 141 can be any article of clothing worn by a person while sleeping when not enrolled in an OSA therapy program. In other words, the personal garment 141 is any wearable article that is not specifically adapted for use during a sleep study, or for diagnosing/monitor/treating a person with OSA.

According to some embodiments, the circuitry 142 can optionally include electronic circuit components configured as a proximity sensor. Embodiments of the proximity sensor are operable to detect the presence of a patient wearing the sensor unit 138 associated with a POSA therapy program. As an example, the proximity sensor can include a capacitive sensor including a transmitter that emits an electrical field. The presence of a part of the patient (e.g., the patient's torso) in the electric field causes a disruption to the electrical field. Such disruptions are detected, allowing the proximity sensor to sense the presence of the patient near the proximity sensor. By comparison, the absence of the patient or other body that would be present if the therapy device was being worn by the patient would result in a relatively undisturbed electric field. So if the patient activates the therapy device and simply places it on a nightstand 146 next to the patient's bed 148 or other surface in an attempt to suggest compliance with the therapy program, any data collected while the therapy device is not properly in use can be scrutinized and optionally filtered from legitimate data (i.e., data captured while the sensor unit 138 is being properly worn by the patient).

As another example, the proximity sensor can include infrared circuitry that emits light in the infrared wavelength spectrum and measures a reflectance signature of the emitted light. According to yet other embodiments, the circuitry of the proximity sensor can optionally include a thermal sensor that detects a heat signature of the patient's body temperature. When being properly worn, the sensor unit 138 can detect an elevated temperature (e.g., greater than typical room temperatures up to eighty (80° F.) degrees Farenheit) indicative of the patient's body temperature. But when placed on a nightstand or otherwise not being worn, the present embodiment of the sensor unit 138 detects a stable, cooler ambient temperature.

Traditional POSA interventions can be disruptive and discourage adherence. In use, the present technology presents a novel positional therapy system that prioritizes patient comfort and self-management. Initially featuring a wearable garment 102 device with removable components in combination with sensor unit 138, it delivers targeted feedback based on sensor-detected sleep position. As therapy progresses, patients gain agency patient customization options (e.g., via a companion mobile app in communication with the transmitter circuitry of the sensor unit 138) and wearable personalization. Data-driven support adjustments, informed by patient feedback and clinician input, empower individuals to adopt lasting habits that improve their sleep and health.

### Stage I

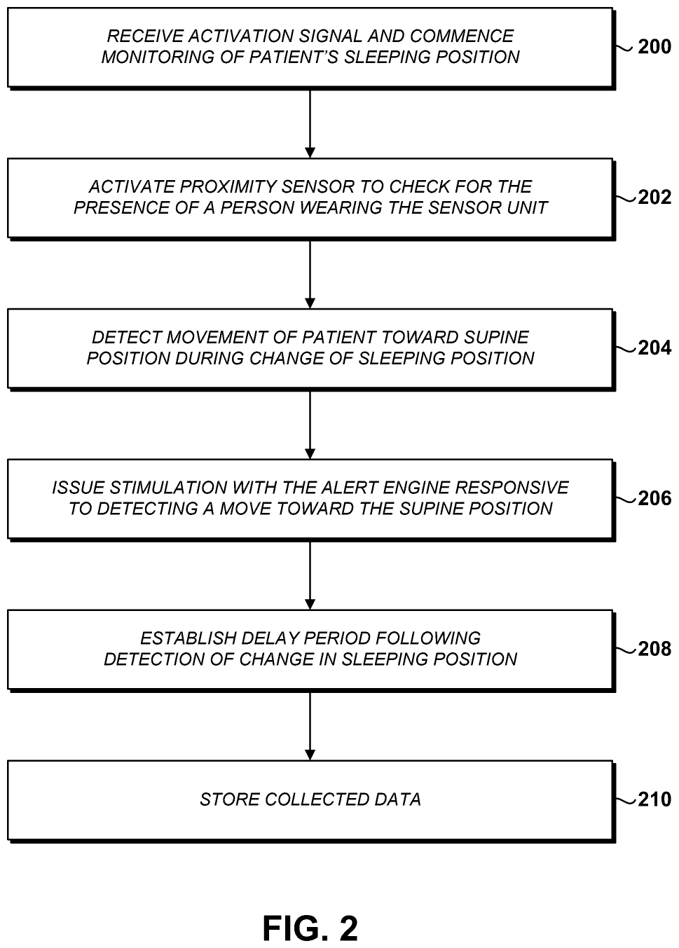

A method of using system 100 as part of a progressive POSA therapy program is described with reference to FIG. 2. Upon being diagnosed with OSA during a lab sleep test, at-home sleep test or other suitable test, or when a patient is suspected to be afflicted with OSA, a patient can be enrolled in Stage I of the program.

In use, Stage I of the progressive POSA therapy program the patient wears garment 102 around their torso with sensor unit 138 disposed within pocket 140, clipped onto back panel 126, or otherwise coupled to garment 102, while sleeping. At block 200, activation signal is received by sensor unit 138 which, in turn, activates monitoring of the patient's sleeping position using the three-position accelerometer circuitry or other position sensor circuitry.

The activation signal can be any input such as the removal of sensor unit 138 (FIG. 1) from a charging cradle, the press of an “On” or “Start” button, the coupling of sensor unit 138 to the patient and/or garment 102, etc. After the therapy device is activated, active monitoring of the patient's sleeping position can begin response to receipt of the activation signal, or can follow expiration of a delay indicated by timing circuitry included in circuitry 142. The delay can be set to allow the patient to begin the process of falling sleep, with the goal of capturing movement representing the patient's actual sleep patterns rather than the patient's tossing and turning prior to falling asleep. According to some embodiments, the delay can be a fixed length of time (e.g., fifteen (15) minutes), or can be a variable length of time to reflect the sleep tendencies of the particular patient wearing the sensor unit 138. For example, the length of the delay can be set by the patient and/or the patient's treating clinician using a companion app 150 running on a mobile device 152 such as a smartphone (e.g., Apple iPhone). According to other embodiments, the length of such a delay can be variable according to an algorithm of the companion app 150. For example, based on data collected using the accelerometer circuitry the algorithm can be used to calculate that the average period of time required for the patient to fall asleep. According to some embodiments, the average length of time for initial movements to stop for at least a defined period of time following receipt of the activation signal (e.g., no significant movements for at least a five (5) minute period occurs, on average, eleven (21) minutes after the activation signal is received).

At block 202 (FIG. 2), the proximity sensor circuitry of sensor unit 138 can be activated to check for the presence of the patient that is supposedly wearing the sensor unit 138. As mentioned elsewhere, the proximity sensor can be configured as a capacitive sensor that detects a disruption of an electromagnetic field, an infrared sensor that is responsive to the patient's heat signature, or other type of sensor that can distinguish between the presence and absence of a human body adjacent to the sensor unit 138. If the presence of a body is not detected by the proximity sensor, a condition that may occur if the patient places the device on a nightstand instead of wearing it, for example, any data collected by the position sensor can be discarded without saving it in the memory, designated as being potentially misleading, etc.

While the sensor unit 138 is being worn by the sleeping patient, the accelerometer circuitry of sensor unit 138 or other movement circuitry detects movement of the patient from one sleeping position to another at block 204 (FIG. 2). For example, if the patient rolls toward the supine position, the accelerometer circuitry will detect the acceleration of the rotating body and trigger the data collection process. In response to sensing movement of the patient toward the supine position, or the patient's arrival in the supine position (within a defined tolerance such as fifteen) (15° degrees in either direction, for example), the alert engine can issue a vibratory and/or audible alert at block 206. For example, the alert engine can include a speaker circuit that emits an audible tone, a haptic or other feedback transducer circuit that issues vibratory alerts, or a combination thereof.

According to some embodiments, circuitry 142 of sensor unit 138 can adjust the stimulation issued in response to the patient sleeping in the supine position. According to such aspects, for patients that regularly respond to vibrations, the magnitude of the vibration from the transducer used for the vibratory alert can be adjusted to a relatively-low setting in an effort to minimize disruptions to the patient's sleep. In contrast, for patients who fail to respond, or only occasionally respond to vibrations, the magnitude of the vibration can be set to a relatively-high level to promote compliance with the therapy by urging the patient to move out of the supine position. Similar adjustments can be made to the volume of audible alerts.

Although the location of the position control member 128 on the garment 102 imposes a physical obstacle to the patient sleeping in the supine position, the vibratory and/or audible alert is issued by the alert engine to “train” the patient to move away from the supine position in response to sensing the alert. Since the position control member 128 forces the patient to move away from the supine position, the patient can subconsciously develop an association between moving away from the supine position with the stimulation from the alert engine. The training goal of Stage I is to eventually trigger a move away from the supine position (e.g., toward a side sleeping position) by the sleeping patient as a natural reaction to the stimulation from the alert engine.

According to some embodiments, when movement of the sleeping patient is detected, the data collection process can optionally utilize timing circuitry of the sensor unit 138 to determine when a delay period following movement has expired at block 208 (FIG. 2). Recording further data collected by the accelerometer circuitry can optionally be suspended during the delay period, or marked as being collected during the delay period, allowing such data to be excluded from further processing. The delay period can be any length of time, but can be chosen to allow the patient to settle into a new position following an in-sleep adjustment. Thus, subsequent movements can be interpreted as actual changes in the patient's sleeping position rather than a momentary adjustment subconsciously made by the patient as part of settling in from the previous adjustment. For example, the timing circuitry can determine when sixteen (16 sec.) seconds have elapsed following movement of the patient.

After the delay period has ended, the proximity sensor of the sensor unit 138 can again check to confirm the presence of the patient. If the patient is detected, data such as the new position of the patient; the date and/or time when the movement triggering the data collection process by the accelerometer occurred; whether any stimulation (e.g., vibratory and/or audible) was issued by the alert engine and, if so, what stimulation was issued by the sensor unit 138 if the patient moved to the supine position; and any other data related to the POSA therapy. If the patient is not detected by the proximity sensor, a condition that may occur if the patient places the device on a nightstand instead of wearing it, any data collected by the position sensor can be discarded without saving it in the memory, designated as being potentially misleading, etc.

The data collected by the position sensor such as the accelerometer, for example, can be stored at block 210 in the local data store of the sensor unit 138 itself, or in a computer-accessible medium provided to a network-connected storage device such as a server, from where the stored data can be retrieved to be displayed by the companion app 150 on the mobile device 152, a network-connected computer terminal, etc. For example, the collected data can be stored initially in the data store of the sensor unit 138 until a connection is established with another storage device. As another example, the data can be initially stored in the data store of the sensor unit 138 temporarily, until the sensor unit 138 is returned to a charging cradle, until a Bluetooth or other network connection is established with a mobile communication device such as the mobile device 152, until a WiFi connection is established, etc. According to such examples, the collected data can be offloaded from the data store of the sensor unit 138 to the other storage location, from where the collected data can be processed and accessed for review by a clinician, caregiver, or other authorized third party through a cloud portal. At least one, a plurality or all of such third parties can optionally access and/or input feedback to the patient via the cloud portal, remotely input updates to operating parameters of the sensor unit 18, update therapy goals, review an overview of patient cohort, or any combination thereof.

The data store can be sized to store data collected over several nights of sleep locally, in a secure format. Once offloaded to another device, or the collected data can be processed using algorithms that present charts, plots, tables, trends, etc. in the collected data indicative of sleep positions, the duration the patient slept in each sleep position, alerts triggered by the alert engine and the timing of such alerts, and other user-configurable feedback options. According to some embodiments, the companion app 150 can be executed to display the collected and/or processed data to the patient, and information pertinent to the progressive POSA therapy program. For example, the companion app 150 can present, and optionally allow for user-modification at least one of:

Goal information such as the percentage of time spent sleeping in the supine position, the total usage time of the sensory unit 138 during the night, etc.;

Visual progress graphs visually indicating trends in the goal information;

Vibratory and/or audible alert customization such as the magnitude/volume of alerts, respectively, the number of alerts issued during a night or other period of time, etc.;

Sleep quality self-logging, which represents the patient's subject feeling on the quality of sleep during a night; and

Clinician messages regarding the progressive POSA therapy program entered via the cloud portal.

### Stage II

The patient can advance to Stage II of the progressive POSA therapy program using sensor unit 138 upon satisfying pre-defined success criteria during Stage I, which can be set by a treating clinician, and optionally related to both sleep position frequency reduction and sensor unit 138 usage time. For example, the patient can progress from Stage I to Stage II if the patient sleeps less than fifteen (21 15%) percent (or other percentage approved by a clinician) of the time spent sleeping in the supine position for at least seventy (70%) percent (or other percentage approved by a clinician) of the previous X number of nights. The value of X can be any integer value established by the clinician, such as between five (5) and one hundred eighty (180) nights, for example.

Stage II can involve many of the processes performed as part of Stage I, but with fewer physical obstacles that interfere with the patient's ability to move into the supine position while sleeping. The advancement from Stage I to Stage II can represent a weaning of the patient from the aids required to physically interfere with the patient's ability to roll into the supine position. Thus, Stage II can be a validation of the “training” of the patient to respond to stimulation from the alert engine while using garment 102 with position control member 128.

For example, sensor unit 138 can be supported adjacent to the patient's torso by garment with the position control member 128 removed from belt 104 or, as shown in FIG. 1, by belt 134. As shown in FIG. 2, Stage II can progress by the activation signal being received by the sensor unit 138 at block 200, and the proximity sensor of sensor unit 138 checking for the presence of the patient adjacent to the sensor unit 138 at block 202. As during Stage I, data collected by the position sensor in the absence of the patient can be discarded without saving it in the memory, designated as being potentially misleading, etc.

During monitoring of the patient's sleeping position, data indicative of the patient changing sleeping positions and moving toward the supine position can be detected by sensor unit 138 at block 204. Responsive to sensing this change in position, the alert engine of the sensor unit 138 can issue stimulation in the form of a vibratory alert and/or an audible alert at block 206. As a result of being urged out of the supine (or near supine) position by position control member 128 while also being exposed to stimulation from the alert engine during Stage I, it is believed the patient will exhibit a learned response to the stimulation from the alert engine, alone, causing the patient to again move away from the supine position. Following the delay period established at block 208, and subject to confirmation that the patient is present as determined by the proximity sensor, the collected data is stored in the data store of sensor unit 138 at block 210, and optionally offloaded thereafter. Data collected in the absence of the patient can be discarded without saving it in the memory, designated as being potentially misleading, etc.

The patient can advance to Stage II of the progressive POSA therapy program using the sensor unit 138 upon satisfying pre-defined success criteria, which can be set by a treating clinician, and optionally related to both sleep position frequency reduction and sensor unit 138 usage time. For example, the patient can progress from Stage I to Stage II if the patient sleeps less than fifteen (21 15%) percent (or other percentage approved by a clinician) of the time spent sleeping in the supine position for at least seventy (70%) percent (or other percentage approved by a clinician) of the previous X number of nights. The value of X can be any integer value established by the clinician, such as between five (5) and one hundred eighty (180) nights, for example.

Stage II can involve many of the processes performed as part of Stage I, but with fewer physical obstacles that interfere with the patient's ability to move into the supine position while sleeping. The advancement from Stage I to Stage II can represent a weaning of the patient from the aids required to physically interfere with the patient's ability to roll into the supine position. Thus, Stage II can be a validation of the “training” of the patient to respond to stimulation from the alert engine while using garment 102 with position control member 128.

For example, sensor unit 138 can be supported adjacent to the patient's torso by garment with the position control member 128 removed from belt 104 or, as shown in FIG. 1, by belt 134. As shown in FIG. 2, Stage II can progress by the activation signal being received by the sensor unit 138 at block 200, and the proximity sensor of sensor unit 138 checking for the presence of the patient adjacent to the sensor unit 138 at block 202. As during Stage I, data collected by the position sensor in the absence of the patient can be discarded without saving it in the memory, designated as being potentially misleading, etc.

During monitoring of the patient's sleeping position, data indicative of the patient changing sleeping positions and moving toward the supine position can be detected by sensor unit 138 at block 204. Responsive to sensing this change in position, the alert engine of the sensor unit 138 can issue stimulation in the form of a vibratory alert and/or an audible alert at block 206. As a result of being urged out of the supine (or near supine) position by position control member 128 while also being exposed to stimulation from the alert engine during Stage I, it is believed the patient will exhibit a learned response to the stimulation from the alert engine, alone, causing the patient to again move away from the supine position. Following the delay period established at block 208, and subject to confirmation that the patient is present as determined by the proximity sensor, the collected data is stored in the data store of sensor unit 138 at block 210, and optionally offloaded thereafter. Data collected in the absence of the patient can be discarded without saving it in the memory, designated as being potentially misleading, etc.

### Stage III

The patient can advance to Stage III of the progressive POSA therapy program using sensor unit 138 upon satisfying pre-defined success criteria during Stage II, which can be set by a treating clinician, and optionally related to both sleep position frequency reduction and sensor unit 138 usage time. However, the criteria for progressing to Stage III may be more demanding than the criteria for advancing to Stage II. For example, the patient can progress from Stage II to Stage III if the patient satisfies all of: (i) sleeps less than eight (21 8%) percent (or other percentage approved by a clinician) of the time in the supine position, (ii) wears sensor unit 138 a prescribed minimum wear time (e.g., six (6) hours per night), and (iii) for at least seventy-five (75%) percent (or other percentage approved by a clinician) of the previous X number of nights. The value of X can be any integer value established by the clinician, such as between five (5) and one hundred eighty (180) nights, for example.

The patient can specify in companion app 150 where on the patient's body sensor unit 138 will be worn while the patient sleeps. For example, the patient may prefer to clip sensor unit 138 to a portion of a waistband at the bottom of the patient's back. Movements detected by the accelerometer circuit can be based on that position to allow the companion app 150 to accurately determine how the patient moved throughout the night while asleep. The companion app 150 can also optionally utilize a user interface that allows the patient to change the location where the therapy device is to be coupled to his/her body, optionally during different Stages. By specifying where sensor unit 138 is to be worn, the present system can relate movements of sensor unit 138 to an adjustment of the patient's sleeping position.

Like Stage II, Stage III can involve many of the processes performed as part of Stage I, but with even fewer physical obstacles that interfere with the patient's ability to move into the supine position while sleeping. The advancement from Stage II to Stage III can represent a further weaning of the patient from the aids required to physically interfere with the patient's ability to roll into the supine position. Thus, Stage III can be a validation of the “training” of the patient to respond to stimulation from the alert engine, without wearing garment 102 with position control member 128.

Unlike Stage II, however, sensor unit 138 is clipped onto or otherwise coupled to personal garment 141 or another article worn by the patient at a location where it provides even less of an obstacle to sleeping in the supine position than the location of sensor unit 138 in Stage II. During Stage II, sensor unit 138 can be supported adjacent to the patient's back by belt 134. Although a compact device, sensor unit 138 could cause the patient to experience some discomfort if sleeping on top of sensor unit 138, in the supine position. During Stage III, however, sensor unit 138 can be clipped onto back neck region 144 of personal garment 141 to be supported adjacent to the back of the patient's neck, or at another location where the patient will experience less discomfort if sleeping in the supine position than during Stage II. However, the location of sensor unit 138 during Stage III will cause the patient to readily receive both a vibratory alert and an audible alert, emitted by the alert engine in close proximity to the patient's ear.

Without going into granular detail for the sake of brevity, as shown in FIG. 2, Stage III can progress by the activation signal being received by the sensor unit 138 at block 200, and the proximity sensor of sensor unit 138 checking for the presence of the patient adjacent to the sensor unit 138 at block 202. Data collected by the position sensor in the absence of the patient can be discarded without saving it in the memory, designated as being potentially misleading, etc.

While monitoring the patient's sleeping position, data indicative of the patient changing sleeping positions and moving toward the supine position can be detected by sensor unit 138 at block 204. Responsive to sensing this change in position, the alert engine of the sensor unit 138 can issue stimulation in the form of a vibratory alert and/or an audible alert at block 206. As a result of continued learning to move out of the supine (or near supine) position responsive to stimulation from the alert engine during Stage II, it is believed the patient will exhibit a learned response to the stimulation from the alert engine, alone, causing the patient to again move away from the supine position. Following the delay period established at block 208, and subject to confirmation that the patient is present as determined by the proximity sensor, the collected data is stored in the data store of sensor unit 138 at block 210, and optionally offloaded thereafter. Data collected in the absence of the patient can be discarded without saving it in the memory, designated as being potentially misleading, etc.

Stage IV

The patient can advance to Stage IV of the progressive POSA therapy program upon satisfying pre-defined success criteria during Stage III, which can be set by a treating clinician, and optionally related to both sleep position frequency reduction and sensor unit 138 usage time. However, the criteria for progressing to Stage IV may be more demanding than the criteria for advancing to Stage III. For example, the patient can progress from Stage III to Stage IV if the patient satisfies all of: (i) sleeps less than five (<5%) percent (or other percentage approved by a clinician) of the time in the supine position, (ii) wears sensor unit 138 a prescribed minimum wear time (e.g., six (6) hours per night), and (iii) for at least eighty (80%) percent (or other percentage approved by a clinician) of the previous X number of nights. The value of X can be any integer value established by the clinician, such as between five (5) and one hundred eighty (180) nights, for example.

During Stage IV, sensor unit 138 is removed from the patient (i.e., not coupled to the patient by garment 102, belt 134, a personal garment 141, or any other article). Instead, a microphone 154 such as that provided to mobile device 152 can be placed adjacent to the patient's bed 148. According to the embodiment shown in FIG. 1, the mobile device 152 running the companion app 150 is placed on nightstand 146 next to the patient's bed 148 and listens for sounds that suggest apneic events. A speaker circuit of the mobile device 152 can issue audible alerts in response to detecting such sounds, with the goal of triggering the patient's learned reaction to move away from the supine position. In each of the embodiments herein, the audible alerts issued during Stages I, II, III and IV can be the same, or at least similar, for the sake of consistency. According to such embodiments, the patient is believed to develop a learned reaction to a given audible alert, and is believed to have been trained to react to that specific audible alert. As a result, the patient is expected to be more responsive to that given audible alert than another audible alert that is different from the audible alert used during one or more of the earlier Stages of the progressive POSA therapy program.

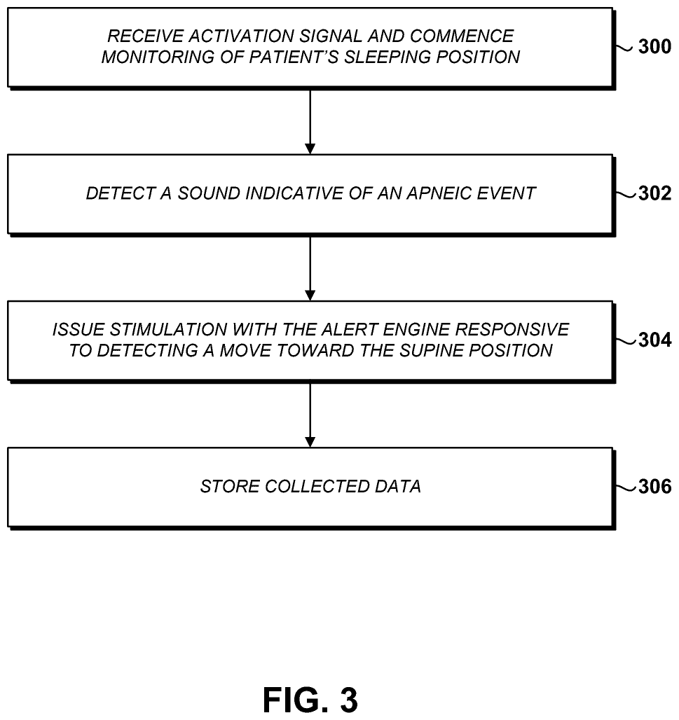

FIG. 3 is a flow diagram schematically depicting a method of encouraging a patient to sleep in a position other than the supine position according to some embodiments of a monitoring system that does not use a sensor physically coupled to the patient or a mechanized respiratory therapy (e.g., CPAP machine). Once the activation signal is received by the companion app 150 at block 300, a timer circuit of the mobile device 152 can establish a delay period during which the patient is assumed to fall asleep. Detection of apneic events can be delayed until after the delay period expires. While the patient is sleeping, microphone 154 picks up sounds indicative of an apneic event at block 302. Responsive to detecting such a sound, an alert engine including at least a speaker circuit provided to the mobile device 152 issues stimulation including an audible alert at block 304. At block 306, data relating to at least one of the following is stored in a data store of the mobile device 152: (i) the date/time the subject sounds were detected, (ii) the alert issued, (iii) the length of time between sounds indicative of an apneic event are detected, and any other relevant information pertaining to the progressive POSA therapy program.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A positional sleep apnea therapy system comprising:
a garment that, in a first configuration, comprises a position control member that interferes with an ability of a patient wearing the garment in the first configuration to sleep in a supine position;
a sensor unit that cooperates with the garment in the first configuration and at least one of: (i) the garment in a second configuration or a second garment that lacks the position control member, and (ii) an article of clothing or an accessory separate from the garment and another garment that is worn by the patient when sleeping, and detects when the patient is sleeping in the supine position, wherein the sensor unit comprises a proximity sensor that detects a presence of the patient adjacent to the sensor unit; and
an alert engine in communication with the sensor unit that issues alerts to the patient in response to the sensor unit detecting that the patient is sleeping in the supine position, wherein the system progresses through at least two stages with adjusted wearable device configurations and alerts.
